(19) United States
(12) Patent Application Publication
(10) Pub. No.: US 2008/0080666 A1
(43) Pub. Date: Apr. 3, 2008

(54) Computed Tomography Imaging With Rotated Detection Modules

(75) Inventor: Raz Carmi, Haifa (IL)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 11/575,660

(22) PCT Filed: Aug. 19, 2005
(86) PCT No.: PCT/IB05/52737

§ 371 Date: Mar. 21, 2007

Related U.S. Application Data
(60) Provisional application No. 60/614,240, filed on Sep. 29, 2004.

(51) Int. Cl. A61B 6/03

(52) U.S. Cl. 378/19

(57) ABSTRACT

A computed tomography imaging apparatus includes a radiation detector (16) having detection modules (18) that are skewed in an axial direction (O_{z}) by a selected angle α. A radiation source (12) provides focal spot modulation at least between two spots (FS1, FS2) to increase a sampling rate transverse to the axial direction (O_{z}) for a more isotropic resolution.

The present application relates to the diagnostic imaging arts. It finds particular application in three-dimensional multi-slice, cone, or wedge beam, more particularly in helical computed tomography imaging, and will be described with particular reference thereto. However, it also finds application in SPECT, PET, and other imaging apparatuses and methods that employ x-ray detectors.

CT scanners typically include an x-ray source and arrays of x-ray detectors secured respectively on diametrically opposite sides of a gantry. During a scan of a patient located within the bore of the gantry, the gantry rotates about a rotation axis while x-rays pass from the focal spot of the x-ray source through the patient to the detectors. An array of projections is simultaneously acquired with dimensions along the direction of gantry rotation, e.g. the Oₓ direction, and along the axial direction, e.g. the O_{z} direction. Increasing resolution in the multi-slice CT scanners with a large axial coverage involves significant costs, as the resolution in such systems depends on the resolution of the detectors and on the rate of data acquisition.

Several cost-effective techniques have been suggested. One technique to increase resolution along the Oₓ direction is to employ a dual focal spot modulation, in which the focal spot is spatially modulated in the Oₓ direction. Another way to increase resolution in the Oₓ direction is by combining opposing rays having a quarter-detector shift. By using both dual focal spot modulation and quarter detector shifting, a factor of four improvement in data sampling in the Oₓ direction can be obtained.

Increasing resolution along the O_{z} direction is important in order to eliminate artifacts in scanners with large axial coverage as well as accurately resolve smaller patterns in the scanned object. However, obtaining data sampling improvement in the O_{z} direction similar to the improvement in the Oₓ direction by a use of focal spot modulation and combining opposing rays is difficult. There is generally no analog to the quarter-detector shift technique for the O_{z} direction, and focal spot modulation in the O_{z} direction is complicated by the x-ray tube anode geometry. For an isotropic x-ray detector array, employing both dual focal spot modulation and quarter-detector shifting in the Oₓ direction with no similar data sampling improvement technique applied in the O_{z} direction results in highly anisotropic data sampling, which is disadvantageous for clinical applications.

One solution for increasing resolution along O_{z} direction is a use of a staggered pixilated array detector. However, the state of the current technology in the area of the detector array manufacture makes the manufacture of the staggered pixilated array a complex and expensive task. The difficulty may be overcome by doubling the cuts of the wafer into pixels, and then combining (on the photodiode) each two small pixels into a one pixel in the desired staggered array. However, due to the additional spacers between the original small pixels, the effective detection area will be reduced by roughly 10-13% and the scanner performance will be reduced. If the whole data measurement system (DMS) is constructed from individual small modules (both along Oₓ and O_{z}), another problem arises. Staggered pixels on any two module-edges (along O_{z}) must be constructed from two separate parts, one from each module (by summing the individual electrical signals). This will require additional electronic channels and may also increase the noise of the combined pixels, potentially resulting in a decrease of the scanner performance.

The present invention contemplates an improved apparatus and method that overcomes the aforementioned limitations and others.

According to one aspect of the present application, a radiographic imaging apparatus is disclosed. A radiation detector has detection modules that are angularly skewed by a prespecified angle greater than 0° and less than 90° in relation to an axial direction. The detection modules are aligned with each other along a transverse direction which is transverse to the axial direction.

According to another aspect, a radiographic imaging method is disclosed. Detection modules of a radiation detector are mounted such that the detection modules are skewed by a prespecified angle greater than 0° and less than 90° in relation to an axial direction. The detection modules are aligned with each other along a transverse direction transverse to the axial direction.

One advantage of the present application resides in increasing resolution in the axial direction.

Another advantage resides in achieving nearly isotropic resolution along Oₓ and O_{z} directions by using standard rectangular modules.

Another advantage resides in increasing resolution at a low cost by a use of standard rectangular detector modules.

Yet another advantage resides in reduced image artifacts and improved image quality.

Numerous additional advantages and benefits will become apparent to those of ordinary skill in the art upon reading the following detailed description of the preferred embodiments.

The invention may take form in various components and arrangements of components, and in various process operations and arrangements of process operations. The drawings are only for the purpose of illustrating preferred embodiments and are not to be construed as limiting the invention.

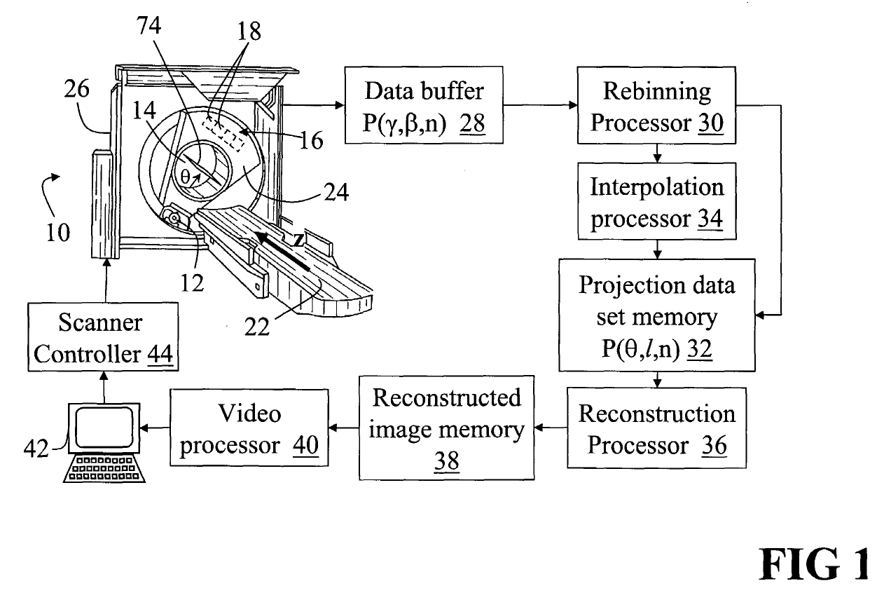

FIG. 1 shows a diagrammatic representation of a computed tomography imaging system;

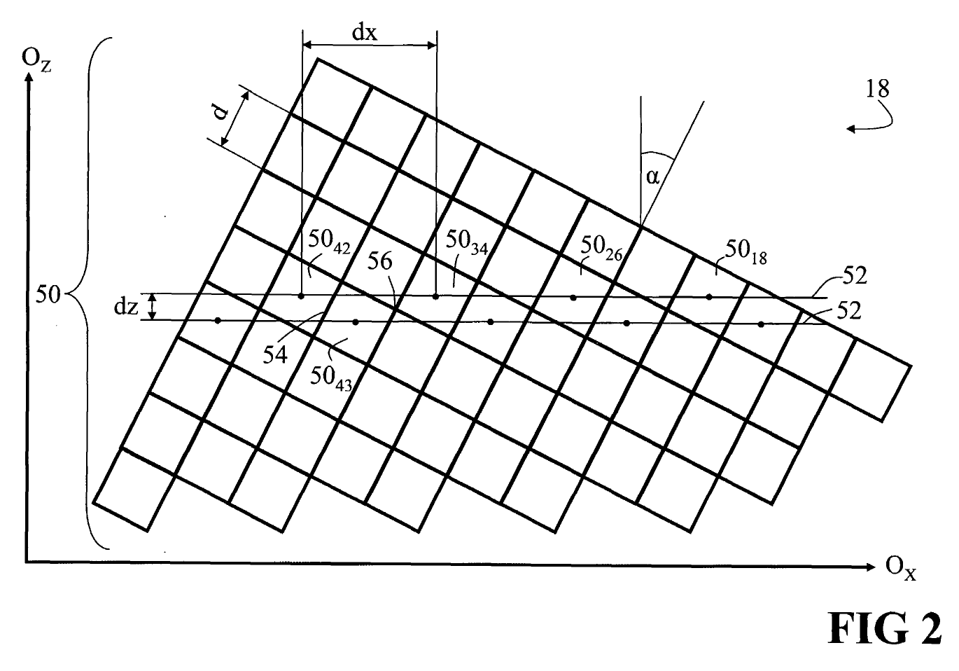

FIG. 2 shows a diagrammatic representation of a portion of the radiation detector module rotated by a first angle;

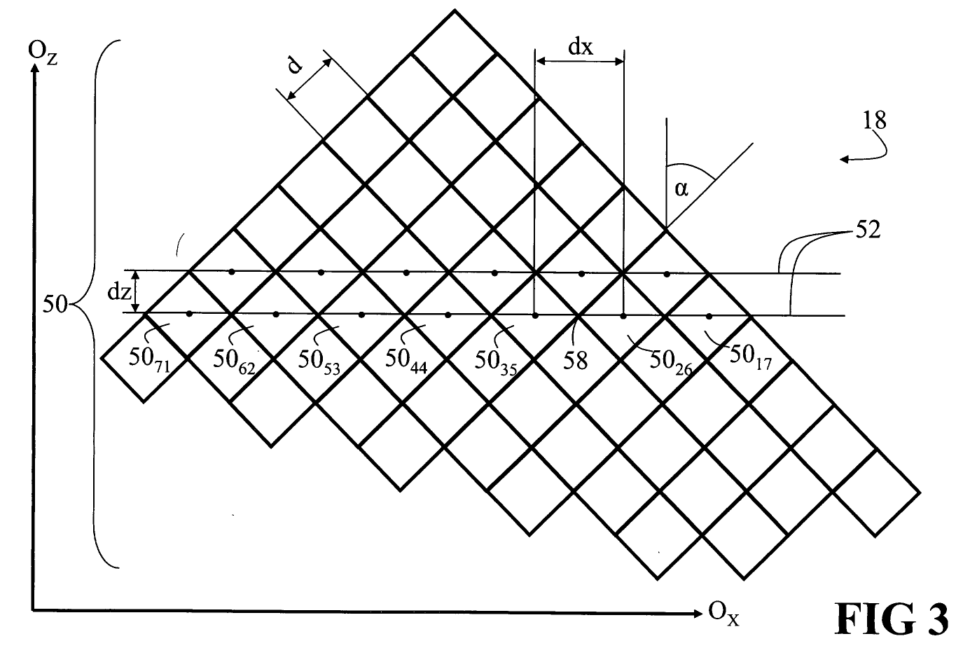

FIG. 3 shows diagrammatic representation of a portion of the radiation detector module rotated by a second angle;

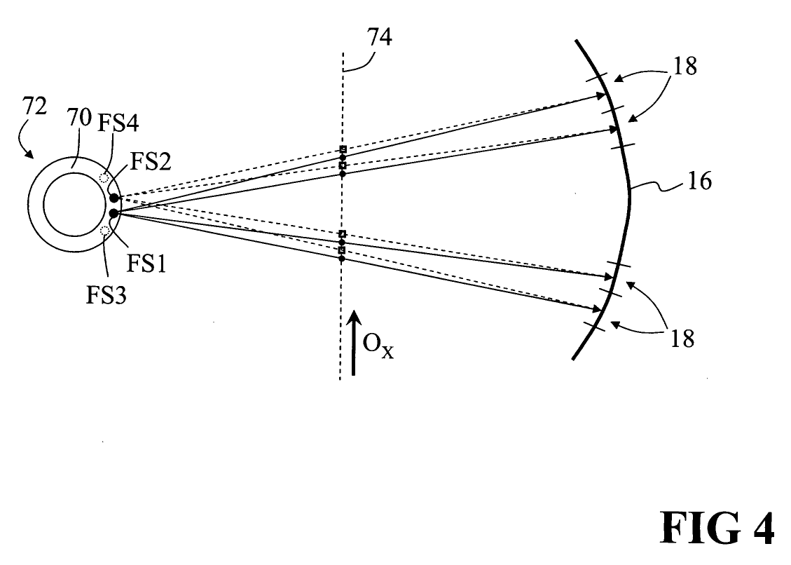

FIG. 4 diagrammatically illustrates the focal spot modulation;

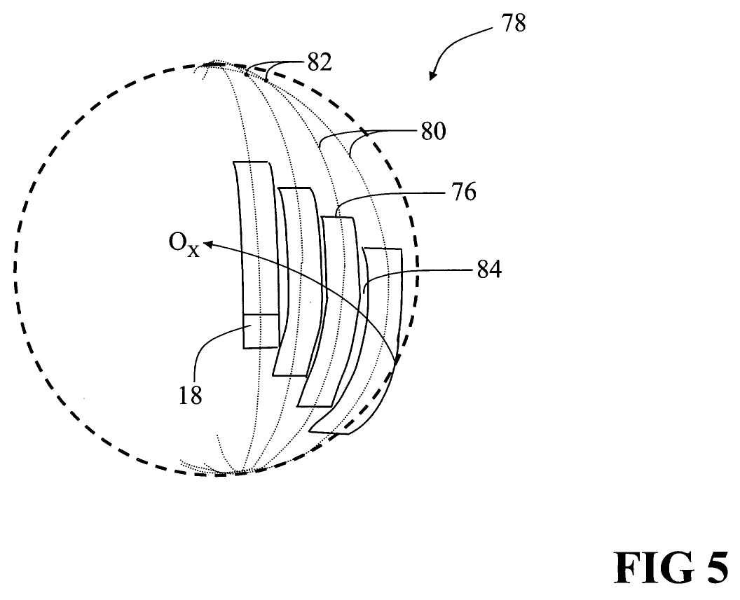

FIG. 5 diagrammatically shows module columns positioned on a spherical surface segment;

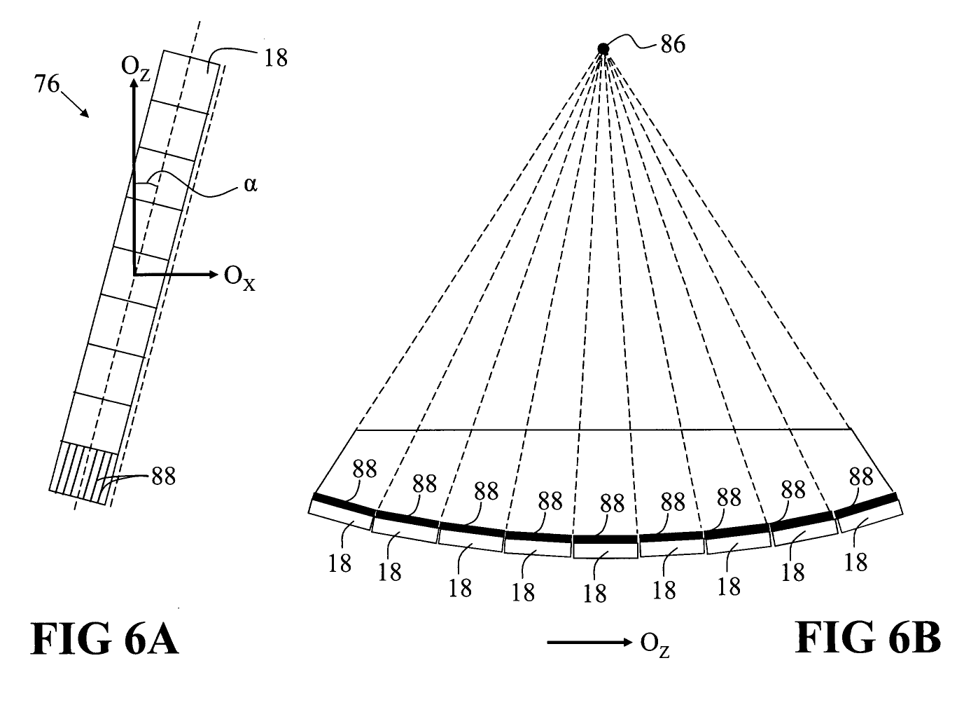

FIG. 6A diagrammatically shows a rotated module column which is straight relative to the focal spot point;

FIG. 6B diagrammatically shows a side view of the detector array;

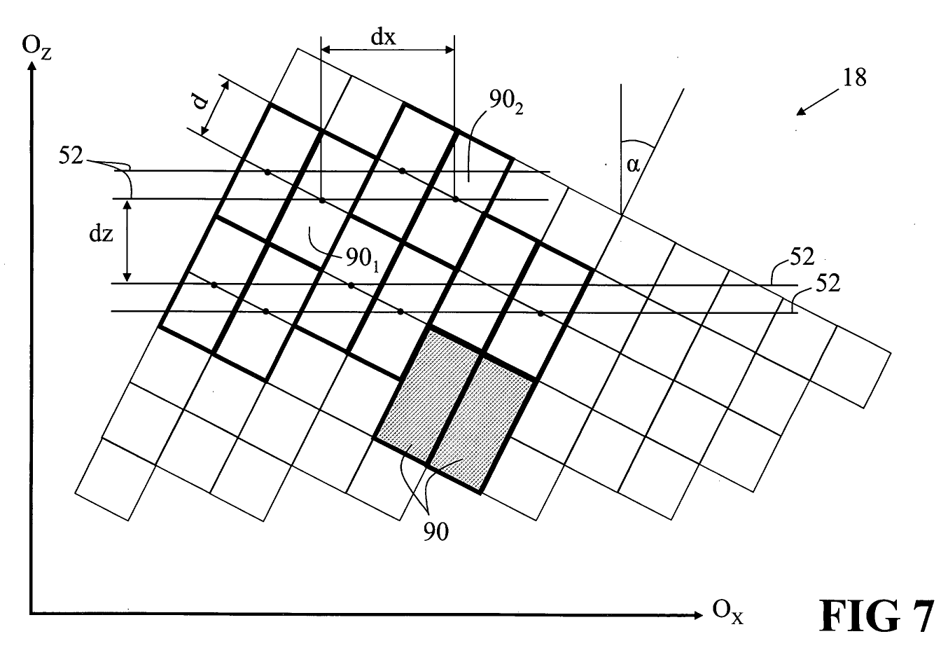

FIG. 7 diagrammatically shows a portion of the rotated radiation detector module in which pixels are combined into detection segments of a first configuration;

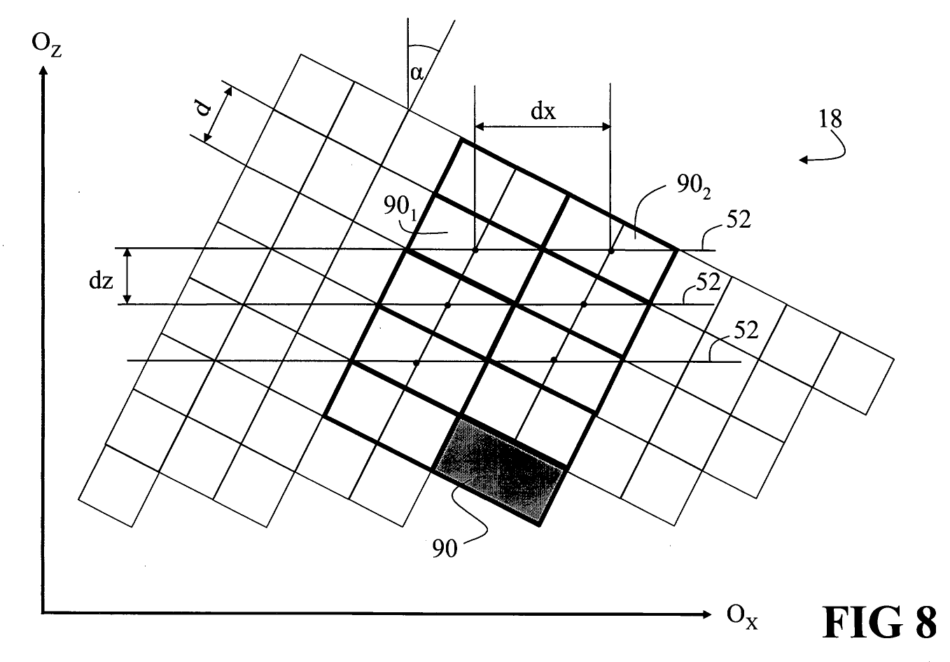

FIG. 8 diagrammatically shows a portion of the rotated radiation detector module in which pixels are combined into detection segments of a second configuration;

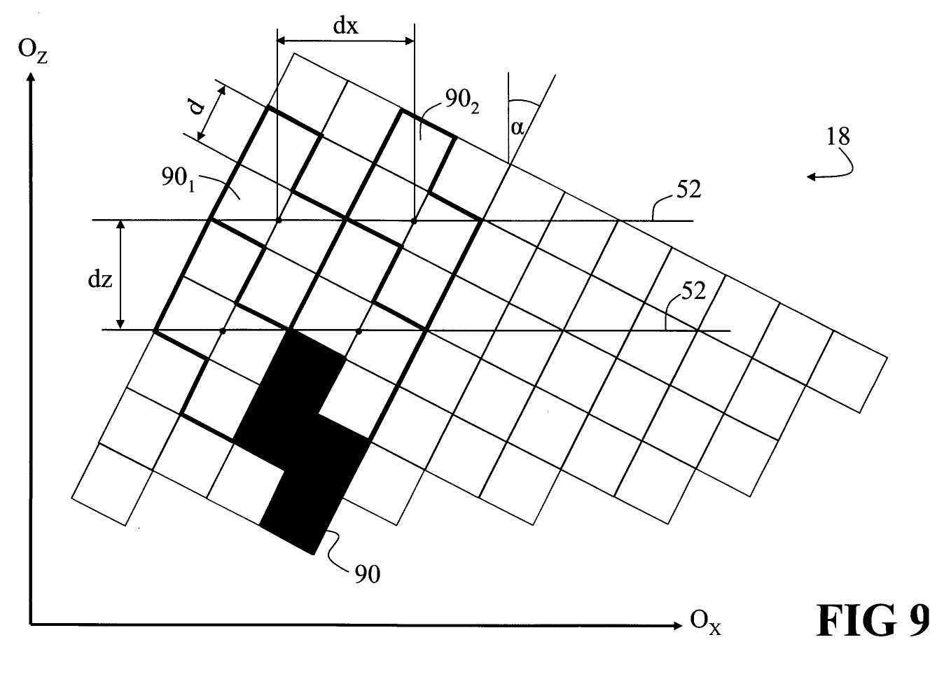

FIG. 9 diagrammatically shows a portion of the rotated radiation detector module in which pixels are combined into detection segments of a third configuration;

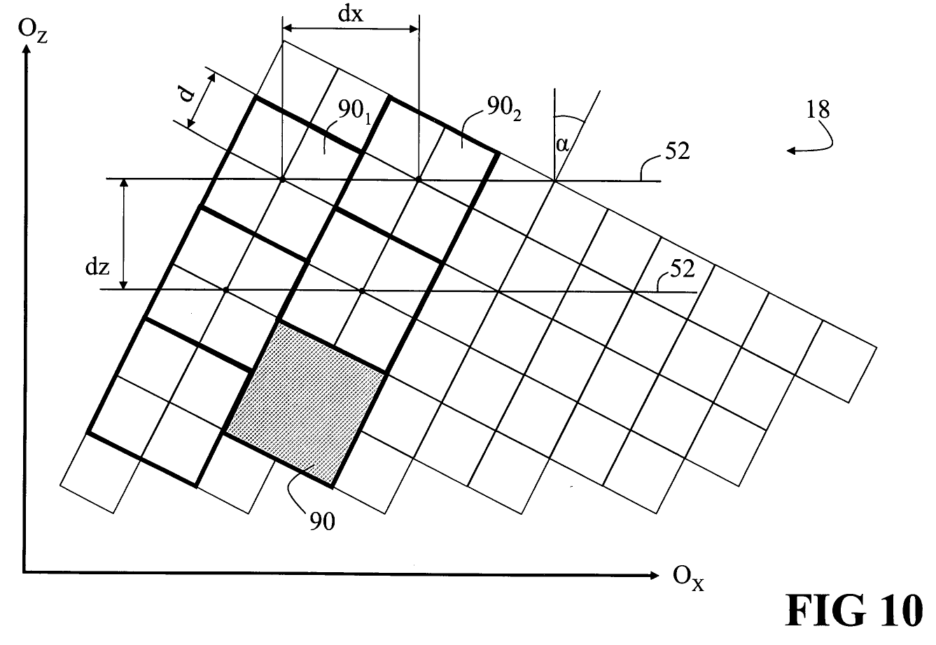

FIG. 10 diagrammatically shows a portion of the rotated radiation detector module in which pixels are combined into detection segments of a fourth configuration;

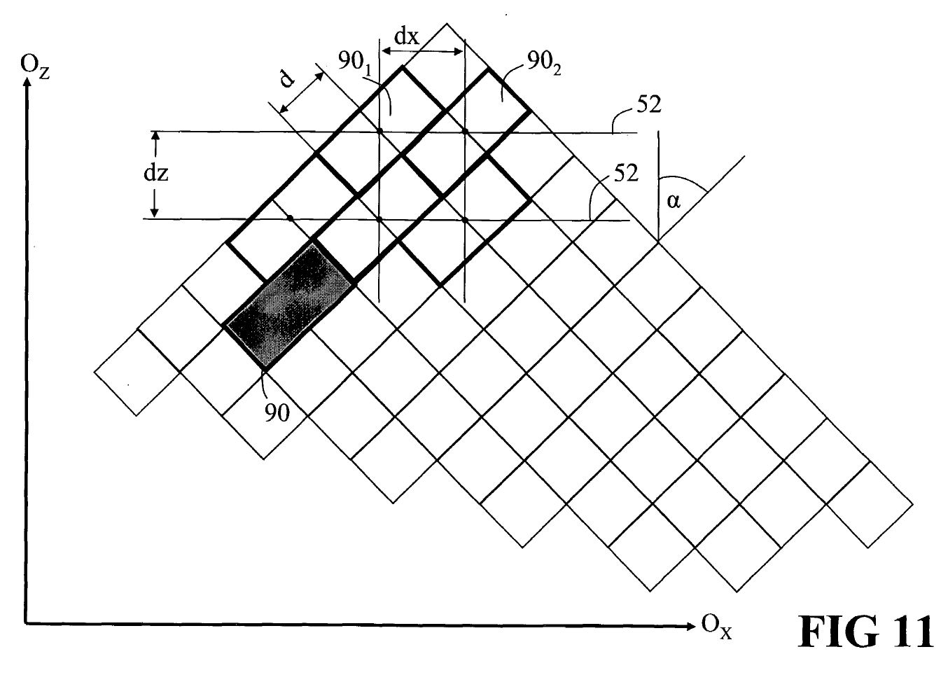

FIG. 11 diagrammatically shows a portion of the rotated radiation detector module in which pixels are combined into detection segments of a fifth configuration; and

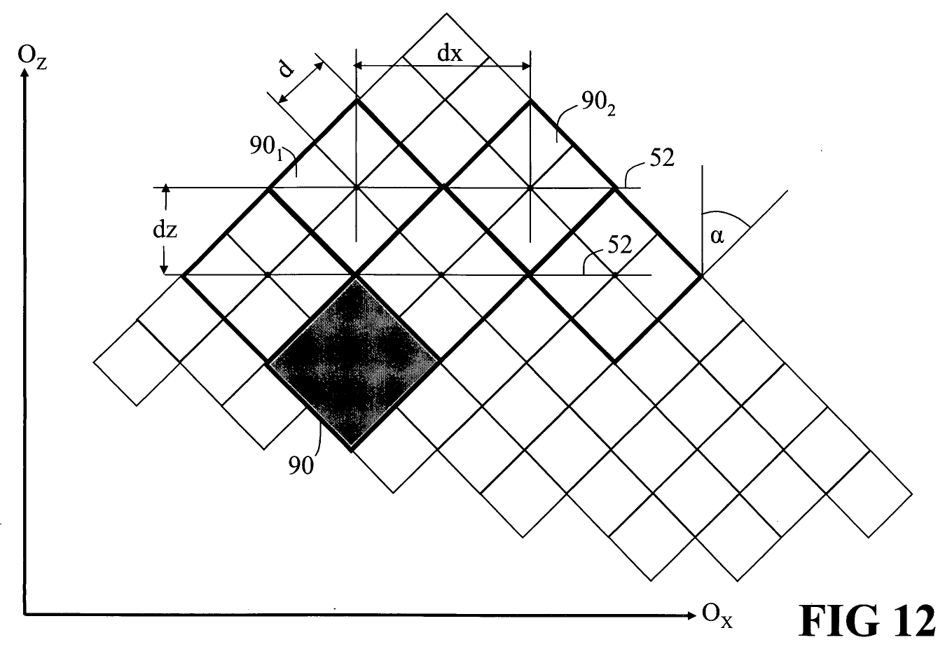

FIG. 12 diagrammatically shows a portion of the rotated radiation detector module in which pixels are combined into detection segments of a sixth configuration.

With reference to FIG. 1, a computed tomography scanner 10 houses or supports a radiation source 12, which in one embodiment is an x-ray source, that projects a radiation beam into an examination region 14 defined by the scanner 10. After passing through the examination region 14, the radiation beam is detected by a two-dimensional radiation detector 16 arranged to detect the radiation beam after passing through the examination region 14. The radiation detector 16 includes a plurality of detection modules or detection elements 18. Each module 18 is rotated about its axis of symmetry by a pre-specified angle α as is discussed in a great detail below. Typically, the x-ray tube produces a diverging x-ray beam having a cone beam, wedge beam, or other beam geometry that expands as it passes through the examination region 14 to substantially fill the area of the radiation detector 16.

An imaging subject is placed on a couch 22 or other support that moves the imaging subject into the examination region 14. The couch 22 is linearly movable along an axial direction O_{z} (designated as a Z-direction in FIG. 1.) The radiation source 12 and the radiation detector 16 are oppositely mounted with respect to the examination region 14 on a rotating gantry 24, such that rotation of the gantry 24 effects revolving of the radiation source 12 about the examination region 14 to provide an angular range of views. The acquired data is referred to as projection data since each detector element detects a signal corresponding to an attenuation line integral taken along a line, narrow cone, or other substantially linear projection extending from the source to the detector element.

In one embodiment, an axial projection data set is acquired with the rotating gantry 24 rotating while the couch 22 is stationary. The axial projection data set includes a plurality of axial slices corresponding to rows or columns of detector elements transverse to the axial or Z-direction. Optionally, additional axial slices are acquired by performing repeated axial scans and moving the couch 22 between each axial scan.

In another embodiment, a helical projection data set is acquired by rotating the gantry 24 simultaneously with continuous linear motion of the couch 22 to produce a helical trajectory of the radiation source 12 around the imaging subject disposed on the couch 22.

During scanning, some portion of the radiation passing along each projection is absorbed by the imaging subject to produce a generally spatially varying attenuation of the radiation. The detector elements of the radiation detector 16 sample the radiation intensities across the radiation beam to generate radiation absorption projection data. As the gantry 24 rotates so that the radiation source 12 revolves around the examination region 14, a plurality of angular views of projection data are acquired, collectively defining a projection data set that is stored in a buffer memory 28.

For a source-focused acquisition geometry in a multi-slice scanner, readings of the attenuation line integrals or projections of the projection data set stored in the buffer memory 28 can be parameterized as P(γ,β,n), where γ is the source angle of the radiation source 12 determined by the position of the rotating gantry 24, β is the angle within the fan (βε[−Φ/2, Φ/2], where Φ is the fan angle), and n is the detector row number in the O_{z} direction. Preferably, a rebinning processor 30 rebins the projection data into a parallel, non-equidistant raster of canonic trans-axial coordinates. The rebinning can be expressed as P(γ,β,n)→P(θ,l,n), where θ parameterizes the projection number that is composed of parallel readings parameterized by 1 which specifies the distance between a reading and the isocenter, and n is the detector row number in the O_{z} direction.

The rebinned parallel ray projection data set P(θ,l,n) is stored in a projection data set memory 32. Optionally, the projection data is interpolated by a interpolation processor 34 into equidistant coordinates or into other desired coordinates spacings before storing the projection data P(θ,l,n) in the projection data set memory 32. A reconstruction processor 36 applies filtered backprojection or another image reconstruction technique to reconstruct the projection data set into one or more reconstructed images that are stored in a reconstructed image memory 38. The reconstructed images are processed by a video processor 40 and displayed on a user interface 42 or is otherwise processed or utilized. In one embodiment, the user interface 42 also enables a radiologist, technician, or other operator to interface with a computed tomography scanner controller 44 to implement a selected axial, helical, or other computed tomography imaging session.

With reference to FIG. 2, a portion of the rectangular detection module 18, e.g. a 16×16 module, is depicted with reference to a detector coordinate directions (Oₓ, O_{z}), in which the O_{z} direction is parallel to the axial or Z-direction of FIG. 1, and Oₓ is transverse to the axial direction or parallel to the rotational direction of the rotating gantry 24. Preferably, each single module 18 includes array of rectangular or square detection pixels 50, as commonly used in CT scanners, which are preferably arranged in a simple rectangular or square matrix n×m. Preferably, the modules have the same dimensions. However, it is contemplated that the modules can have different dimensions. Each module 18 is rotated to align the centers of the exemplary pixels 50₄₂, 50₃₄, 50₂₆, 50₁₈ along an associate row 52 parallel to the rotational direction Oₓ. The exemplary pixels 50₄₂, 50₃₄, 50₂₆, 50₁₈ are selected to have a first aligned pixel to share a common side with a third pixel which lies along a neighboring row 52 parallel to Oₓ; and a second aligned pixel to share a common corner with the third pixel. E.g., the first aligned pixel 50₄₂ shares a common side 54 with the third pixel 50₄₃; and the second aligned pixel 50₃₄ shares a common corner 56 with the third pixel 50₄₃. In such a configuration with square pixels, the angle of rotation a is equal to arctan (0.5) or approximately to 26.565°. Of course, when the pixels are not square, the angle of rotation depends on the pixels dimensions. The rows 52 are equally spaced along the axial direction O_{z}; and the centers of the aligned pixels are equally spaced along the axis of rotation Oₓ. If a width d of the pixel 50 is defined as unity or 1 (in arbitrary units), the distance dz between the rows 52 is inversely proportional to the resolution along the axial direction O_{z} and is equal to 1/√5. The distance dx between the centers of the two pixels aligned along the row 52 is inversely proportional to the resolution along the rotational direction Oₓ and is equal to √5.

With reference to FIG. 4, the resolution or sampling rate along the rotational direction Oₓ is improved by a factor of two by using focal spot modulation of the radiation source 12 in the Oₓ direction. The focal spot is shifted between two positions FS1 and FS2 on a beveled surface 70 of an anode 72 of the radiation source 12. The separation of the focal spots FS1, FS2 at the anode 72 is selected to shift the projections at a meridian plane 74 (shown in FIG. 1) by a distance proportional to one-half of the distance dx between the centers of the two pixels aligned along the row 52. Filled circles on the meridian plane 74 indicate samples acquired using the focal spot FS1, and open squares on the meridian plane 74 indicate samples acquired using the focal spot FS2.

The sampling rate along the rotational direction Oₓ can be alternatively improved by a factor of three or four by using three or four focal spot modulation of the radiation source 12 in the Oₓ direction. The possible four focal spots are shown in phantom by positions FS3 and FS4 in FIG. 4. The separation of the focal spots at the anode 72 is then selected to shift the projections at a meridian plane 74 by a distance proportional correspondingly to one-half, one-third or one-fourth of the distance dx between the centers of the two pixels aligned along the row 52.

With reference again to FIG. 2, to achieve a nearly isotropic resolution, the focal spot modulation with four points is preferably employed. E.g., if the distance dx between the centers of the two pixels along the row 52 is equal to √5, the ratio of the sampling distance is equal to

R=(dx/4)/dz=(√5/4)/(1/√5)=1.25,

which provides nearly isotropic resolution.

In one embodiment, it is more advantageous to choose a combination other than one which provides the isotropic resolution to achieve other objectives. For example, in one embodiment, it is more advantageous to use focal spot modulation of three positions rather than four. In such system, the resolution in the Oₓ direction is relatively less improved, but the maximal rotation time is less limited than in the system where the four position focal spot modulation is used.

With reference to FIG. 3, the detection module 18 is rotated to align the centers of the exemplary pixels 50₇₁, 50₆₂, 50₅₃, 50₄₄, 50₃₅, 50₂₆, 50₁₇ along an associated row 52 parallel to the rotational direction Oₓ similarly to the embodiment of FIG. 2. The pixels 50₇₁, 50₆₂, 50₅₃, 50₄₄, 50₃₅, 50₂₆, 50₁₇ are selected to have a first aligned pixel to share a common corner with the second aligned pixel. E.g., the first aligned pixel 50₃₅ shares the common corner 58 with the second aligned pixel 50₂₆. In the configuration of FIG. 3, the angle of rotation α is equal to 45°. Of course, when the pixels are not square, the angle of rotation depends on the pixels dimensions. The rows 52 are equally spaced along the axial direction O_{z}; and the centers of the pixels aligned along the rows 52 are equally spaced along the axis of rotation Oₓ. The distance dz between the rows 52 defines the resolution along the axial direction O_{z} and is equal to 1/√2. The distance dx between the centers of the two pixels lying along the row 52 defines the resolution along the rotational direction Oₓ and is equal to √2.

With continuing reference to FIG. 3 and reference again to FIG. 4, the sampling rate along the rotational direction Oₓ is preferably improved by a factor of two, three or four by a use of a focal spot modulation with two, three or four different positions along the axis of rotation Oₓ. If the focal spot modulation with four points is used, e.g. sampling distance=dx/4=√2/4, the ratio of the sampling distance is

R=(dx/4)/dz=(√2/4)/(1/√2)=0.5

With reference to FIG. 5, in a large area cone beam embodiment, the detection modules 18 are merged into module columns 76 which are assembled on the DMS cradle in a configuration in which the DMS global shape is curved preferably along both Oₓ and O_{z} directions, such that each module 18 faces directly the focal spot mean position (not shown) which is located in the center of a sphere 78. The modules 18 are rotated on the DMS cradle by the angle a in relationship to the axial direction O_{z} to provide a continuous coverage across the entire DMS. The number of modules 18 in each column 76 is determined by the module size and the required coverage along the axial direction O_{z}. A centerline 80 of each column 76 is tangential to the sphere 78, and cross points 82 of two centerlines 80 are different for each two neighboring columns 76. Notably, the modules 18 are not curved.

Optionally, the DMS shape is not curved along O_{z} direction, e.g. for wedge beams, although the curvature of the DMS along the axial direction O_{z} is highly favorable with respect to the large coverage along the axial direction O_{z}; mainly due to the requirement to align modules toward the focal spot position in order to eliminate problems regarding the use of two-dimensional anti-scatter grid which is preferably used to improve image quality. However, it is contemplated that a standard one-dimensional ASG might be used. Due to the curvature of the DMS surface along Oₓ and O_{z} directions, small spaces 84 between the module columns 76 are introduced. The width of the spaces 84 is of the order of 50 μm for the DMS which covers about 80 mm at the isocenter (e.g. 128 slices). It should be noted that when the DMS with a large axial coverage is constructed from “non-rotated” modules, it is highly probable that the curvatures would be introduced along both Oₓ and O_{z} directions in order to eliminate problems regarding the use of two-dimensional anti-scatter grid. In this case, the spaces between modules would be of similar order compared to the spaces in the rotated module configuration.

With reference to FIGS. 6A and 6B, the modules 16 are tiled along the rotated module symmetry axis in order to create a straight detector column 76 relative to the focal spot point of view 86. An anti-scatter grid 88 is oriented parallel to the modules 18 orientation. A single long anti-scatter grid (ASG) unit can be assembled on the module column 76. If small separate ASG units are in use, the tiling along the column 76 is not mandatory. In the arrangement of the module column 76, the lamellas of the long ASG (one-dimensional or two-dimensional grid) do not require any mechanical twisting, thus a standard ASG manufacturing technique can be used.

With reference to FIGS. 7-10, the CT scanner includes options to electronically or by other means combine two or more adjacent pixels 50 into a detection segment 90. The module 18 is rotated by the angle of rotation α, which in a case of square pixels is preferably equal to arctan (0.5), to align the centers of the detection segments 90₁, 90₂, . . . , 90ₙ along associated rows 52 parallel to the rotational direction Oₓ.

With continuing reference to FIG. 7, combinations of two adjacent pixels form the detection segments 90. In this configuration, the rows 52 are not equally spaced along the axial direction O_{z}, but the centers of the detection segments 90₁, 90₂, . . . , 90ₙ are equally spaced along the axis of rotation Oₓ. If the width d of the pixel 50 is assumed to be 1 (in arbitrary units), the maximum distance dz between the rows 52 is roughly inversely proportional to the resolution along O_{z} and, is equal to 3/√5. The distance dx between the centers of the detection segments 90₁, 90₂, . . . , 90ₙ along the row 52 is inversely proportional to the resolution along Oₓ and is equal to √5. The resolution or sampling rate along the rotational direction Oₓ might be improved by a use of a focal spot modulation by a factor of two, three or four positions along the rotational axis Oₓ.

With reference again to FIG. 8, combinations of two adjacent pixels form the detection segments 90. In this configuration, the rows 52 are equally spaced along the axial direction O_{z}, and centers of the detection segments 90₁, 90₂, . . . , 90ₙ are equally spaced along the axis of rotation Oₓ. The distance dz between the rows 52 is related to the resolution along the axial direction O_{z} and is equal to 2/√5. The distance dx between the centers of the detection segments 90₁, 90₂, . . . , 90ₙ along the row 52 is related to the resolution along the rotational direction Oₓ and is equal to √5. The resolution or sampling rate along the rotational direction Oₓ might be improved by a factor of two, three or four by a use of a focal spot modulation with two, three or four different positions along the axis of rotation Oₓ.

With reference again to FIG. 9, combinations of four adjacent pixels 50 form the detection segments 90. The rows 52 are equally spaced along the axial direction O_{z}; and the centers of the detection segments 90₁, 90₂, . . . , 90ₙ are equally spaced along the axis of rotation Oₓ. The distance dz between the rows 52 is related to the resolution along the axial direction O_{z} and is equal to 4/√5. The distance dx between the centers of the detection segments 90₁, 90₂, . . . , 90ₙ along the row 52 is related to the resolution along the rotational direction Oₓ and is equal to √5. The resolution or sampling rate along the rotational direction Oₓ might be improved by a factor of two, three or four by a use of a focal spot modulation with two, three or four different positions along the axis of rotation Oₓ.

With reference again to FIG. 10, combinations of four adjacent pixels 50 form rectangular detection segments 90. The rows 52 are equally spaced along the axial direction O_{z}; and the centers of the detection segments 90₁, 90₂, . . . , 90 ₙ are equally spaced along the axis of rotation Oₓ. The distance dz between the rows 52 is related to the resolution along the axial direction O_{z} and is equal to 4/√5. The distance dx between the centers of the detection segments 90₁, 90₂, . . . , 90ₙ along the row 52 is related to the resolution along the rotational direction Oₓ and is equal to √5. The resolution or sampling rate along the rotational direction Oₓ might be improved by a factor of two, three or four by using a focal spot modulation with two, three or four different positions along the axis of rotation Oₓ.

With reference to FIGS. 11-12, the module 18 is rotated by the angle of rotation α, which is preferably equal to 45° (in case of square pixels), to align the centers of the detection segments 90₁, 90₂, . . . , 90ₙ along associated rows 52 parallel to the rotational direction Oₓ.

With continuing reference to FIG. 11, combinations of two adjacent pixels form the detection segments 90. In this configuration, the rows 52 are equally spaced along the axial direction O_{z}, and centers of the detection segments 90₁, 90₂, . . . , 90ₙ are equally spaced along the axis of rotation Oₓ. The distance dz between the rows 52 is related to the resolution along the axial direction O_{z} and is equal to √2. The distance dx between the centers of the detection segments 90₁, 90₂, . . . , 90ₙ along the row 52 is related to the resolution along the rotational direction Oₓ and is equal to √2. The resolution or sampling rate along the rotational direction Oₓ might be improved by a factor of two, three or four by a use of a focal spot modulation with two, three or four different positions along the axis of rotation Oₓ.

With reference again to FIG. 12, combinations of four adjacent pixels 50 form rectangular detection segments 90. The rows 52 are equally spaced along the axial direction O_{z}; and the centers of the detection segments 90₁, 90₂, . . . , 90ₙ are equally spaced along the axis of rotation Oₓ The distance dz between the rows 52 is related to the resolution along the axial direction O_{z} and is equal to √2. The distance dx between the centers of the detection segments 90₁, 90₂, . . . , 90ₙ along the row 52 is related to the resolution along the rotational direction Oₓ and is equal to 2√2. The resolution or sampling rate along the rotational direction Oₓ might be improved by a factor of two, three or four by using a focal spot modulation with two, three or four different positions along the axis of rotation Oₓ.

In another embodiment, a nuclear (e.g. SPECT or PET) camera is provided. The x-ray source is a radiopharmaceutical which is injected into the subject. The heads have solid state detectors of the constructions described above.

In another embodiment, a projection x-ray device is provided with an angularly displaced solid state detector as described above.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

1. A radiographic imaging apparatus comprising:
a radiation detector having detection modules that are angularly skewed by a prespecified angle greater than 0° and less than 90° in relation to an axial direction and aligned with each other along a transverse direction transverse to the axial direction.

2. The radiographic imaging apparatus as set forth in claim 1, wherein each module includes a plurality of pixels which pixels are aligned to place center points of the pixels on straight rows parallel to the transverse direction.

3. The radiographic imaging apparatus as set forth in claim 2, wherein a first pixel aligned in a first row shares only a common corner with a second adjacent pixel aligned in the first row.

4. The radiographic imaging apparatus as set forth in claim 2, wherein a first pixel aligned in a first row shares a common side with a third pixel aligned in a second row parallel to the first row, and a second pixel aligned in the first row shares a common corner with the third pixel.

5. The radiographic imaging apparatus as set forth in claim 2, further including:
a radiation source providing focal spot modulation that increases a sampling rate parallel to the transverse direction.

6. The radiographic imaging apparatus as set forth in claim 5, wherein the focal spot modulation produces one of two, three and four projections separated by one of corresponding first, second or third distance each corresponding distance is being proportional to a distance between the centers of the aligned pixels in the transverse direction.

7. The radiographic imaging apparatus as set forth in claim 6, wherein the angle is equal to arctan and the focal spot modulation produces four projections to achieve a substantially isotropic resolution.

8. The radiographic imaging apparatus as set forth in claim 6, wherein the angle is equal to 45° and the focal spot modulation produces one of two and three projections.

9. The radiographic imaging apparatus as set forth in claim 1, wherein each detection module includes:
a rectangular array of detector elements which are aligned with first and second orthogonal axes, one of the first and second orthogonal axes being angularly skewed from the axial direction by the prespecified angle.

10. The radiographic image apparatus as set forth in claim 9, wherein the prespecified angle is one of 26.565° and 45°.

11. The radiographic imaging apparatus as set forth in claim 9, further including:
a radiation source with focal spot modulation that increases a sampling rate in the transverse direction.

12. The radiographic imaging apparatus as set forth in claim 1, further including:
a radiation source;
a gantry for rotating the source around the axial direction;
a means for moving an associated imaging subject parallel to the axial direction.

13. A radiographic imaging method comprising:
mounting detection modules of a radiation detector skewed by a prespecified angle greater than 0° and less than 90° in relation to an axial direction; and
aligning the detection modules with each other along a transverse direction transverse to the axial direction.

14. The method as set forth in claim 13, further including:
mounting a radiation source to rotate with the detector; and
modulating the radiation source between at least two focal spot positions spaced apart transverse to the axial direction.

15. The method as set forth in claim 14, further including:
selecting a number of the focal spot positions based on a resolution along the axial direction to achieve more isotropic resolution.

16. The method as set forth in claim 13, wherein the detection modules include a plurality of pixels and wherein the mounting step includes:
aligning center points of the pixels to coincide with straight rows transverse to the axial direction.

17. The method as set forth in claim 13, wherein the detection modules each include a rectangular array of detector elements which are aligned along first and second orthogonal axes, and wherein the mounting step includes:
aligning each detection module such that one of the first and second orthogonal axes are skewed from the axial direction by the prespecified angle.

18. The method as set forth in claim 17, further including:
modulating a radiation source irradiating the detector between at least two focal spot positions spaced apart transverse to the axial direction.

19. The method as set forth in claim 13, wherein the mounting step includes:
arranging the detection modules into columns; and
placing the columns on a surface which is curved along the transverse direction and along the axial direction.

20. A radiographic imaging apparatus for performing the method of claim 13.
